(19) United States
(12) Patent Application Publication
(10) Pub. No.: US 2025/0166778 A1
(43) Pub. Date: May 22, 2025

(54) METHODS AND SYSTEMS WITH INTEGRATED VASCULAR ASSESSMENT

(71) Applicant: Wound Pros Technology, Inc., Los Angeles, CA (US)

(72) Inventors: Bill J. Releford, Los Angeles, CA (US); Daniel Lee Hallman, Los Angeles, CA (US)

(73) Assignee: Wound Pros Technology, Inc., Los Angeles, CA (US)

(21) Appl. No.: 18/952,675

(22) Filed: Nov. 19, 2024

Related U.S. Application Data
(60) Provisional application No. 63/601,020, filed on Nov. 20, 2023.

(51) Int. Cl. G16H 20/30; A61B 5/026; G16H 10/60; G16H 15/00

(52) U.S. Cl. CPC G16H 20/30; A61B 5/0261; G16H 10/60; G16H 15/00

(57) ABSTRACT

This disclosure provides methods and systems for providing a patient-specific wound care plan for a patient based on vascular assessment. The disclosed methods and systems integrate a vascular diagnostic tool with Electronic Health Record (EHR) systems, leveraging advanced automation and artificial intelligence (AI) to generate patient-specific treatment plans based on real-time vascular assessments. This integration addresses the need for seamless data transfer and enhanced clinical efficiency in wound care, particularly for patients requiring consistent vascular monitoring.

### CROSS-REFERENCE TO RELATED APPLICATIONS

This application claims priority under 35 U.S.C. § 119(e) to U.S. Provisional Patent Application No. 63/601,020, filed Nov. 20, 2023. The foregoing application is incorporated by reference herein in its entirety.

### FIELD OF THE INVENTION

This invention relates generally to methods and systems with integrated vascular assessment and automated generation of patient-specific wound care plans.

### BACKGROUND OF THE INVENTION

Current healthcare systems face significant challenges in the accuracy, efficiency, and consistency of vascular assessment and treatment planning for wound care. Manual data entry often leads to errors, delays in diagnosis, and inconsistencies in treatment plans, compromising patient outcomes. For example, due to poor integration of existing diagnostic tools with electronic medical record (EMR) (or electronic health record (EHR)) systems, it is challenging for a clinician to assess vascular exams and prepare personalized treatment plans in patients' records. Thus, there exists a need for methods and systems that integrate diagnostic tools with EMR or EHR systems. There is a critical need for a solution that can streamline the integration of diagnostic tools, ensuring seamless and error-free data flow into EHR systems to support clinical decisions and enhance patient care.

### SUMMARY OF THE INVENTION

This disclosure provides a method for providing a patient-specific wound care plan for a patient based on vascular assessment. In some embodiments, the method comprises: (a) receiving patient vascular assessment data of the patient, wherein the patient vascular assessment data comprises vascular history data, vascular symptom data, and vascular examination data; (b) processing and loading the patient vascular assessment data to an electronic health record system; (c) analyzing the patient vascular assessment data by a trained model; (d) generating, by the trained model, a patient-specific wound care plan for the patient based on the vascular history data, the vascular symptom data, and the vascular examination data; (e) populating the patient-specific wound care plan within the electronic health record system; and (f) presenting the patient vascular assessment data and the patient-specific wound care plan on an interface accessible to a health care provider.

In some embodiments, the vascular history data comprises one or more of: age 50 years or older, African American ethnicity, currently on blood thinners, diabetes, elevated homocysteine levels, family history of vascular disease, history of amputations, high blood pressure, high cholesterol, history of gangrene, history of heart disease, inflammatory conditions, kidney disease, male gender, obesity or overweight, open wound, physical inactivity, tobacco use history, stroke, and no prior vascular health history.

In some embodiments, the vascular symptom data comprises one or more of: experienced pain, cramping, or discomfort in leg(s) while walking or during physical activity; leg pain at rest or during the night; change in leg color, temperature, and/or texture of feet or legs; non-healing sores, wounds, or ulcers on the feet or legs; swelling in leg(s), ankle(s), and/or feet; numbness, tingling, or sensations of pins and needles in your feet and/or legs; and none.

In some embodiments, the vascular physical examination data comprises one or more of: visible varicose or spider veins noted on the leg(s) and/or feet; palpable pedal pulses; non-palpable pedal pulses; hair loss and skin changes in texture on your legs and/or feet; and none.

In some embodiments, the patient vascular assessment data comprises real-time vascular assessment data.

In some embodiments, the patient vascular assessment data is obtained from a QuantaFlo system that is integrated with the electronic health record system.

In some embodiments, the patient vascular assessment data is obtained from Optical Character Recognition (OCR) of handwritten and printed records.

In some embodiments, the patient vascular assessment data is obtained from a third-party vascular report.

In some embodiments, the method comprises converting data from the QuantaFlo system into HL7-compliant messages to be integrated into the electronic health record system.

In some embodiments, the method comprises converting data from the QuantaFlo system into HL7-compliant messages by a Mirth interface.

In some embodiments, the data is encrypted.

In some embodiments, the interface comprises a clinical dashboard accessible to a clinician.

In some embodiments, the clinical dashboard allows interactive modifications to the patient-specific wound care plan by the clinician.

In some embodiments, the trained model comprises a machine learning model. In some embodiments, the machine learning model comprises a supervised or unsupervised machine learning model.

In some embodiments, the machine learning model comprises Deep Learning algorithm, Logistic Regression, Naive Bayes, Support Vector Machine, Decision Tree, Random Forest, Gradient Boosting, Regularizing Gradient Boosting, K-Nearest Neighbors, a continuous regression approach, Ridge Regression, Kernel Ridge Regression, Support Vector Regression, deep learning approach, Neural Networks, Convolutional Neural Network (CNNs), Recurrent Neural Networks (RNNs), Gated Recurrent Units (GRUs), Long Short Term Memory Networks (LSTMs), Generative Models, Generative Adversarial Networks (GANs), Deep Belief Networks (DBNs), Feedforward Neural Networks, Autoencoders, Variational Autoencoders, Normalizing Flow Models, Deniosing Diffusion Probabilistic Models (DDPMs), Score Based Generative Models (SGMs), Radial Basis Function Networks (RBFNs), Multilayer Perceptrons (MLPs), Stochastic Neural Networks, or a combination thereof.

In another aspect, this disclosure also provides a system for providing a patient-specific wound care plan for a patient based on vascular assessment, comprising one or more processors configured to implement the method as described herein.

In yet another aspect, this disclosure additionally provides a non-transitory computer-readable medium comprising instructions that, when executed by one or more processors, cause the one or more processors to perform the method as described herein.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features is not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 shows an example workflow illustrating data flow from QuantaFlo to RITA Electronic Health Record (EHR) systems.

FIG. 2 shows a clinical dashboard interface mockup.

FIG. 3 shows an example security and compliance flowchart illustrating data encryption and access control measures.

### DETAILED DESCRIPTION OF THE INVENTION

Current healthcare systems face significant challenges in the accuracy, efficiency, and consistency of vascular assessment and treatment planning for wound care. To address these challenges, this disclosure provides methods and systems for providing a patient-specific wound care plan for a patient based on vascular assessment. The disclosed methods and systems integrate a vascular diagnostic tool with Electronic Health Record (EHR) systems, leveraging advanced automation and artificial intelligence (AI) to generate patient-specific treatment plans based on real-time vascular assessments. The integration addresses the need for seamless data transfer and enhanced clinical efficiency in wound care, particularly for patients requiring consistent vascular monitoring. The system includes data captured through optical character recognition (OCR), HL7-compliant communication via Mirth integration, and/or automated workflows within the EHR environment, optimizing the accuracy, speed, and quality of patient care.

In some embodiments, the method comprises: (a) receiving patient vascular assessment data of the patient, wherein the patient vascular assessment data comprises vascular history data, vascular symptom data, and vascular examination data; (b) processing and loading the patient vascular assessment data to an electronic health record system; (c) analyzing the patient vascular assessment data by a trained model; (d) generating, by the trained model, a patient-specific wound care plan for the patient based on the vascular history data, the vascular symptom data, and the vascular examination data; (e) populating the patient-specific wound care plan within the electronic health record system; and (f) presenting the patient vascular assessment data and the patient-specific wound care plan on an interface accessible to a health care provider.

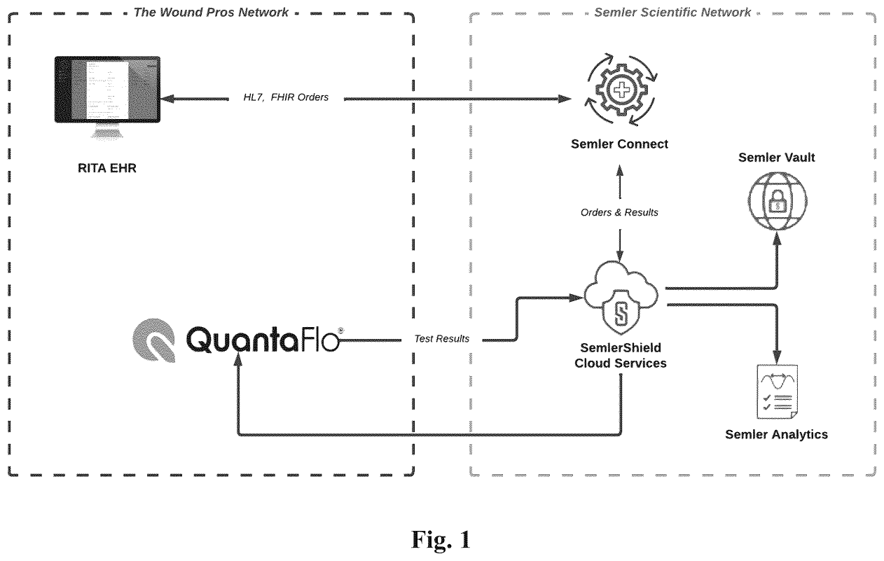

In some embodiments, as shown in FIG. 1, the system may perform data capture and interpretation through QuantaFlo integration, optical character recognition (OCR), and/or third-party vascular report integration. QuantaFlo provides real-time vascular assessment. QuantaFlo is a non-invasive, FDA-approved diagnostic test designed to assess blood flow in the arms and legs, helping to detect peripheral arterial disease (PAD). The test works by placing sensors on a patient's fingers and toes to measure circulation and identify differences in blood flow. It can be conducted in both clinical settings or at home, providing results in just minutes. Utilizing digital volume plethysmography and infrared sensors, QuantaFlo accurately measures blood flow in the extremities, enabling the early detection of PAD. The system captures output directly from QuantaFlo, enabling automated, accurate input into the EHR system. OCR technology scans and digitizes handwritten and printed inputs to reduce data entry errors, feeding this information directly into the EHR. The EHR system enables users to upload third-party vascular reports directly into the patient's chart, analyze the data within the chart, and generate a customized treatment plan tailored to the patient's specific vascular health needs.

In some embodiments, the system may include a data communication layer that integrates Health Level Seven (HL7)/Mirth. Health Level Seven (HL7) is a standards-developing organization that creates international standards for exchanging health information. HLTs standards are used to transfer clinical and administrative health data between applications, with the goal of improving patient outcomes and health system performance. Mirth is a cross-platform HL7 interface that implements bi-directional HL7 messages between systems and apps over multiple devices. It is utilized to build more accessible, more secure, and economical interoperable mechanisms. The Mirth interface in the disclosed systems converts QuantaFlo data into HL7-compliant messages for seamless integration into EHR systems. Mirth serves as an intermediary, ensuring compatibility and data integrity.

In some embodiments, the system also includes an automated clinical workflow. The automated clinical workflows may include AI-based analysis and recommendations, automated treatment plans, and/or AI-powered analysis and recommendations for third-party vascular reports. AI-based analysis and recommendations are enabled through algorithms that analyze real-time vascular assessment data (e.g., QuantaFlo data) to generate patient-specific vascular treatment recommendations. Automated treatment plans can be generated based on the AI analysis, and the system populates the vascular plan of care within the EHR, providing a tailored, ready-to-use treatment plan for clinicians. AI-powered analysis and recommendations for third-party vascular reports are enabled through algorithms that analyze real-time QuantaFlo data to provide patient-specific vascular treatment recommendations, enhancing the precision and effectiveness of care. In some embodiments, AI algorithms process vascular assessment data (e.g., QuantaFlo data), generating a customized vascular plan of care. This automation ensures that each patient receives a treatment plan tailored to their unique vascular profile, which clinicians can further customize as needed. The vascular plan of care includes immediate intervention recommendations, follow-up schedules, and referral prompts, aiding clinicians in delivering timely and personalized care.

In some embodiments, the system may include a interface, such as a clinical dashboard. The clinical dashboard provides an intuitive interface displaying real-time vascular assessment results and suggested treatment plans, accessible to clinicians and administrative staff. Through this interface, clinicians can adjust the treatment plan as necessary, with all modifications logged and updated in real-time, ensuring traceability.

In some embodiments, the system can have regulatory and compliance features, including HIPAA compliance and FDA guidelines for diagnostic tools. In some embodiments, all patient data is encrypted and stored in compliance with HIPAA standards, with access controls to ensure patient confidentiality. In some embodiments, the system adheres to regulatory standards for medical devices and diagnostic technologies, ensuring compliance with FDA guidelines for patient safety.

In some embodiments, the system employs two factor authentication (2FA) for user access and Transport Layer Security (TLS) encryption for data transfers. Additionally, all data stored within the EHR is encrypted, meeting HIPAA requirements and ensuring regulatory compliance.

The disclosed methods and systems have several important advantages, including enhanced diagnostic accuracy, reduced clinician workload, regulatory adherence and data security, and scalability across clinical settings. For example, OCR and QuantaFlo integration improve diagnostic accuracy, significantly reducing the margin for error in capturing vascular assessment data. By automating the data entry and treatment planning process, clinicians can focus on patient interaction, improving quality of care and patient satisfaction. HIPAA and FDA-compliant features safeguard patient data and meet industry standards, making the system suitable for use in diverse healthcare settings. In addition, the system's HL7 compatibility allows easy integration with various EHR systems, making it adaptable for home health, hospice, skilled nursing facilities (SNFs), and other clinical settings. The customizable user interface can scale for large databases, allowing implementation in multiple healthcare settings.

This integrated diagnostic tool for vascular assessment fills a critical gap in wound care by combining real-time data capture with AI-driven treatment recommendations and secure EHR integration. The system's innovative use of OCR, HL7-compliant data communication, and AI for plan generation positions it as a unique solution for efficient and effective wound care, providing a high level of accuracy, security, and adaptability. By automating complex clinical workflows and adhering to regulatory standards, the invention significantly enhances patient outcomes and clinician efficiency, establishing a strong basis for intellectual property protection.

In some embodiments, the patient vascular assessment data comprises vascular history data, vascular symptom data, and vascular examination data.

In some embodiments, the vascular history data comprises one or more of: age 50 years or older, African American ethnicity, currently on blood thinner, diabetes, elevated homocysteine levels, family history of vascular disease, history of amputations, high blood pressure, high cholesterol, history of gangrene, history of heart disease, inflammatory conditions, kidney disease, male gender, obesity or overweight, open wound, physical inactivity, tobacco use history, stroke, and no prior vascular health history.

Vascular History refers to a patient's past and current medical conditions, lifestyle factors, and family history that are related to the health and function of the vascular system. This information helps healthcare providers assess a patient's risk for vascular diseases, such as peripheral artery disease (PAD), coronary artery disease (CAD), stroke, and other complications related to poor circulation.

In some embodiments, the vascular history data comprises the following parameters:

1. Age 50 Years or Older—Patients in this age group have a higher risk of vascular complications.
a. Treatment Plan:
   i. Routine screening for vascular health, including assessments for PAD, hypertension, and cholesterol levels.
   ii. Encourage lifestyle modifications, such as regular physical activity, balanced nutrition, and smoking cessation.
   iii. Annual check-ups to monitor for early signs of vascular disease and comorbidities.

2. African American Ethnicity—Known to have a higher predisposition to vascular disease.
a. Treatment Plan:
   i. Regular screening for hypertension, diabetes, and PAD, as African Americans are at higher risk for these conditions.
   ii. Focus on early intervention strategies and lifestyle modifications, including diet, exercise, and medication adherence.
   iii. Encourage health education and self-management practices to mitigate risks.

3. Currently on Blood Thinner—Use of anticoagulants for managing blood flow and preventing clots.
a. Treatment Plan:
   i. Monitor INR (international normalized ratio) levels regularly to ensure appropriate blood thinning effect.
   ii. Assess the need for compression stockings to prevent swelling and improve circulation.
   iii. Educate patients on the risks of bleeding and the importance of regular monitoring.
   iv. Collaborate with a vascular specialist to assess for any bleeding complications or side effects of anticoagulants.

4. Diabetes—Chronic condition affecting blood vessels and increasing risk of vascular disease.
a. Treatment Plan:
   i. Tight glycemic control through diet, exercise, and medications (e.g., insulin or oral agents).
   ii. Regular monitoring of blood glucose levels and HbA1c.
   iii. Annual screening for diabetic complications, including PAD and neuropathy.
   iv. Encourage wound care management, especially for diabetic foot ulcers.

5. Elevated Homocysteine Levels—Indicator of potential cardiovascular and vascular health issues.
a. Treatment Plan:
   i. Consider folic acid, vitamin B6, and B12 supplementation to lower homocysteine levels.
   ii. Monitor cardiovascular health closely and consider using statins or other appropriate medications to manage overall cardiovascular risk.
   iii. Promote heart-healthy lifestyle choices, including a balanced diet, regular exercise, and smoking cessation.

6. Family History of Vascular Disease—Includes relatives with conditions like PAD, CAD, or other vascular issues.
a. Treatment Plan:
   i. Early screening for PAD, CAD, and other vascular diseases based on family history.
   ii. Aggressive management of modifiable risk factors (e.g., blood pressure, cholesterol, smoking cessation).
   iii. Encourage genetic counseling if indicated.
   iv. Regular follow-up with vascular health assessments.

7. History of Amputations—Previous limb loss due to vascular complications or other causes.
a. Treatment Plan:
   i. Regular monitoring of vascular status, especially in the remaining limb(s), to prevent further complications.
   ii. Assess for signs of infection or poor circulation in the affected area.
   iii. Encourage rehabilitation and physical therapy to improve mobility and quality of life.
   iv. Provide psychological support for coping with the impact of amputation.

8. High Blood Pressure—Hypertension contributing to blood vessel damage and increased vascular risk.
a. Treatment Plan:
   i. Initiate or adjust antihypertensive medications to achieve target blood pressure (e.g., ACE inhibitors, ARBs, beta-blockers).
   ii. Lifestyle modifications, including reducing sodium intake, regular exercise, and stress management.
   iii. Routine blood pressure monitoring and adjustments as necessary.
   iv. Screen for end-organ damage, such as kidney or heart disease.

9. High Cholesterol—Elevated lipid levels that can lead to plaque build-up in blood vessels.
a. Treatment Plan:
   i. Prescribe statins or other lipid-lowering medications as appropriate.
   ii. Encourage a heart-healthy diet, including increased fiber intake, healthy fats, and limited saturated fats.
   iii. Regular lipid profile testing to monitor cholesterol levels.
   iv. Promote exercise and weight management.

10. History of Gangrene—Past tissue death due to inadequate blood supply, indicating severe vascular issues.
a. Treatment Plan:
   i. Close monitoring for signs of ischemia and possible recurrence of gangrene.
   ii. Regular vascular assessments, including ABI, ultrasound, or angiography.
   iii. Ensure proper wound care, infection control, and appropriate footwear.
   iv. Smoking cessation and optimal management of comorbid conditions (e.g., diabetes, hypertension).

11. History of Heart Disease—Includes conditions such as coronary artery disease or heart attack.
a. Treatment Plan:
   i. Regular cardiovascular screenings, including EKG, echocardiograms, and stress tests.
   ii. Aggressive management of cardiovascular risk factors (e.g., cholesterol, blood pressure, lifestyle).
   iii. Consideration of antiplatelet therapy (e.g., aspirin) to reduce the risk of further events.
   iv. Cardiac rehabilitation, if indicated, to improve heart function and overall health.

12. Inflammatory Conditions—Chronic inflammation affecting vascular health, e.g., arthritis.
a. Treatment Plan:
   i. Manage underlying inflammatory condition (e.g., rheumatoid arthritis) with appropriate immunosuppressive medications.
   ii. Regular monitoring for potential vascular complications related to inflammation.
   iii. Encourage physical activity and stretching to maintain joint function and vascular health.
   iv. Consider physical therapy for management of pain and inflammation.

13. Kidney Disease—Reduced kidney function that can exacerbate vascular health complications.
a. Treatment Plan:
   i. Close monitoring of kidney function (e.g., serum creatinine, GFR).
   ii. Control blood pressure and blood sugar to prevent progression of kidney disease.
   iii. Assess for peripheral edema and signs of vascular insufficiency.
   iv. Referral to nephrology for more specialized care and dialysis, if needed.

14. Male Gender—Male patients have a statistically higher risk of vascular disease.
a. Treatment Plan:
   i. More frequent screening for vascular disease, especially PAD and CAD, as males are at higher risk.
   ii. Early intervention for modifiable risk factors (e.g., smoking, obesity, high blood pressure).
   iii. Encourage healthy lifestyle changes to reduce overall cardiovascular and vascular risk.

15. Obesity or Overweight—Excess weight linked to higher vascular risk, affecting blood pressure and vessel health.
a. Treatment Plan:
   i. Initiate weight management programs that include diet modification, exercise, and possibly medications.
   ii. Screen for related conditions like hypertension, diabetes, and sleep apnea.
   iii. Encourage lifestyle changes focused on sustainable weight loss and healthy eating habits.
   iv. Regular monitoring of comorbid conditions.

16. Open Wound—Presence of a wound that may have impaired healing due to poor circulation.
a. Treatment Plan:
   i. Aggressive wound care, including debridement, infection control, and use of appropriate dressings.
   ii. Monitor for signs of poor circulation that may impede wound healing.
   iii. Referral to vascular specialists if arterial insufficiency or venous issues are suspected.
   iv. Regular follow-up to ensure proper wound healing and prevent complications.

17. Physical Inactivity—Sedentary lifestyle associated with a higher risk of vascular disease.
a. Treatment Plan:
   i. Develop an individualized exercise program to improve circulation and overall cardiovascular health.
   ii. Encourage regular walking, swimming, or cycling to improve vascular function.
   iii. Provide education on the importance of staying active to reduce vascular disease risks.
   iv. Monitor progress and adjust the exercise plan as needed to match the patient's capacity.

18. Tobacco Use History—History of smoking, a known risk factor for vascular and cardiovascular disease.
a. Treatment Plan:
   i. Provide smoking cessation resources (e.g., counseling, nicotine replacement therapy).
   ii. Screen for related vascular conditions like PAD, CAD, and lung disease.
   iii. Educate the patient on the detrimental effects of smoking on vascular health.
   iv. Follow-up to ensure continued cessation and monitor for improvements in circulation.

19. Stroke—History of cerebrovascular events indicating compromised vascular health.
a. Treatment Plan:
   i. Regular follow-up with neurologists and vascular specialists to monitor for further cerebrovascular issues.
   ii. Antiplatelet therapy (e.g., aspirin) to reduce the risk of recurrence.
   iii. Address modifiable risk factors such as hypertension, diabetes, and cholesterol.
   iv. Rehabilitation programs to assist in recovery and prevent future strokes.

20. None—Option to indicate no prior vascular health history.
a. Treatment Plan:
   i. Provide education on maintaining vascular health and preventing future vascular issues.
   ii. Routine screenings for risk factors and early detection of vascular conditions.
   iii. Encourage healthy lifestyle practices, including balanced diet, exercise, and regular check-ups.

In some embodiments, the vascular symptom data comprises one or more of: experienced pain, cramping, or discomfort in leg(s) while walking or during physical activity; leg pain at rest or during the night; change in leg color, temperature, and/or texture of feet or legs; non-healing sores, wounds, or ulcers on the feet or legs; swelling in leg(s), ankle(s), and/or feet; numbness, tingling, or sensations of pins and needles in your feet and/or legs; and none.

Symptom history data refers to a patient's reported experiences of symptoms that may indicate underlying vascular conditions. This data helps healthcare providers assess the severity and type of vascular disease a patient may have. It includes detailed descriptions of any pain, discomfort, changes in appearance, or other physical manifestations related to the vascular system. Each symptom can point to different vascular conditions and help guide diagnostic and treatment decisions.

In some embodiments, the vascular symptom data comprises the following parameters:

1. Experienced pain, cramping, or discomfort in leg(s) while walking or during physical activity
a. Purpose: This symptom is testing for claudication, a condition caused by reduced blood flow to the muscles during activity. It may indicate peripheral artery disease (PAD), where narrowed arteries reduce circulation, leading to pain or cramping in the legs during exertion.
b. Treatment Plan:
   i. Diagnosis: Perform an ABI (Ankle-Brachial Index) test, doppler ultrasound, or angiography to confirm PAD.
   ii. Medications: Initiate antiplatelet therapy (e.g., aspirin or clopidogrel) to reduce clot formation and improve blood flow.
   iii. Lifestyle Modifications: Recommend a supervised exercise program (e.g., walking) to improve circulation and build collateral circulation.
   iv. Smoking Cessation: Counsel and provide resources for smoking cessation, as smoking exacerbates PAD.
   v. Referral: Refer to a vascular surgeon or interventional cardiologist for further assessment and possible angioplasty or stenting if the disease is advanced.

2. Leg pain at rest or during the night
a. Purpose: This symptom is testing for ischemic pain, which occurs when blood flow is severely restricted, often indicating advanced PAD or critical limb ischemia (CLI). Pain at rest or while lying down suggests that the legs are not receiving sufficient blood supply even when at rest, which can be a sign of severe vascular disease.
b. Treatment Plan:
   i. Diagnosis: Perform diagnostic imaging (e.g., duplex ultrasound, CTA, MRA) to assess for critical limb ischemia (CLI).
   ii. Medications: Use vasodilators (e.g., cilostazol) to improve blood flow and pain relief. Pain management with appropriate analgesics (e.g., opioids, if necessary) may be required.
   iii. Lifestyle Modifications: Instruct the patient to elevate the legs to encourage venous return and reduce swelling.
   iv. Surgical Intervention: Consider revascularization procedures (angioplasty, bypass surgery) if the condition is severe and limiting daily activities.
   v. Referral: Refer to a vascular specialist for consideration of advanced therapeutic options such as stenting or surgery.

3. Change in leg color, temperature, and/or texture of feet or legs
a. Purpose: This symptom relates to circulatory issues such as poor perfusion. A change in color (pale or bluish skin), temperature (cooler than usual), or texture (dry, shiny, or scaly skin) of the feet or legs can indicate arterial insufficiency, where the arteries fail to supply enough oxygenated blood to tissues.
b. Treatment Plan:
   i. Diagnosis: Perform an ABI, duplex ultrasound, and skin perfusion pressure testing to confirm the presence of arterial insufficiency.
   ii. Medications: Prescribe medications to improve circulation (e.g., pentoxifylline or cilostazol).
   iii. Wound Care: If changes in texture (dry, scaly skin) are present, ensure the use of moisturizers and specialized foot care to prevent ulcers.
   iv. Lifestyle Modifications: Recommend regular movement and elevation of the legs to enhance blood flow.
   v. Referral: Refer to a vascular specialist for further evaluation and consideration of revascularization if appropriate.

4. Non-healing sores, wounds, or ulcers on the feet or legs
a. Purpose: This symptom is testing for wound healing complications, which may be associated with poor circulation, diabetes, or other vascular conditions. Non-healing ulcers, particularly on the feet or legs, can indicate chronic venous insufficiency (CVI) or diabetic foot ulcers, both of which can be caused or worsened by vascular disease.
b. Treatment Plan:
   i. Diagnosis: Perform a thorough wound assessment, including culture for infection, and ABI testing to assess perfusion.
   ii. Wound Care: Initiate appropriate wound care protocols, including debridement, moist wound healing dressings, and infection control measures.
   iii. Medications: Use systemic antibiotics if infection is present. Consider growth factor or cellular tissue products (CTPs) for non-healing ulcers.
   iv. Compression Therapy: For venous ulcers, initiate compression therapy to reduce edema and improve venous return.
   v. Referral: Refer to a wound care specialist or vascular surgeon for advanced management if healing does not occur.

5. Swelling in leg(s), ankle(s), and/or feet
a. Purpose: This symptom is checking for edema, which can be a sign of venous insufficiency, where blood has difficulty returning to the heart from the lower extremities. It could also indicate heart failure, kidney disease, or deep vein thrombosis (DVT), where the veins in the legs become congested with blood or fluid.
b. Treatment Plan:
   i. Diagnosis: Perform diagnostic tests, including a venous duplex ultrasound to assess for venous insufficiency or deep vein thrombosis (DVT). Kidney and heart function should also be evaluated (e.g., serum creatinine, echocardiogram).
   ii. Medications: Consider diuretics to manage fluid retention, especially if heart failure or kidney disease is contributing to edema.
   iii. Compression Therapy: Initiate compression stockings or bandages to help with venous return and reduce swelling.
   iv. Lifestyle Modifications: Encourage leg elevation, weight management, and regular physical activity to improve circulation.
   v. Referral: If there is a concern for DVT, refer to a specialist for anticoagulation therapy or further management.

6. Numbness, tingling, or sensations of “pins and needles” in your feet and/or legs
a. Purpose: This symptom is testing for neuropathy, which often occurs in combination with vascular conditions such as diabetes. Numbness or tingling can indicate poor circulation and nerve damage due to reduced blood flow to the nerves, common in diabetic neuropathy or other systemic vascular diseases.
b. Treatment Plan:
   i. Diagnosis: Perform a thorough neurological exam, including sensory testing, and assess blood glucose levels and HbA1c to rule out diabetes-related neuropathy.
   ii. Medications: Consider the use of medications for neuropathic pain (e.g., gabapentin, pregabalin, or duloxetine).
   iii. Lifestyle Modifications: Recommend regular foot inspections, proper footwear, and good foot hygiene to prevent injury and infection.
   iv. Blood Sugar Control: Tight glycemic control through diet, exercise, and medications is essential for managing diabetes-related neuropathy.
   v. Referral: Refer to a neurologist or pain specialist for further management if symptoms persist or worsen.

7. None
a. Purpose: This option is included for patients who do not experience any of the listed vascular symptoms, helping to rule out significant vascular issues or identify asymptomatic individuals.
b. Treatment Plan:
   i. Prevention and Health Maintenance: Encourage routine health check-ups and screenings (e.g., annual blood pressure checks, cholesterol testing, and diabetes screening).
   ii. Lifestyle Modifications: Emphasize a healthy diet, regular exercise, and smoking cessation to reduce the risk of developing vascular diseases in the future.
   iii. Patient Education: Educate the patient about recognizing early signs of vascular problems, including changes in leg health, to promote early detection and intervention if symptoms arise.

In some embodiments, the vascular physical examination data comprises one or more of: visible varicose or spider veins noted on the leg(s) and/or feet; palpable pedal pulses; non-palpable pedal pulses; hair loss and skin changes in texture on your legs and/or feet; and none.

Vascular physical examination data refers to the observable signs and physical findings during an examination that may indicate the presence or absence of vascular conditions. These parameters help healthcare providers assess circulation and detect early signs of vascular disease, allowing for timely intervention or further diagnostic testing.

In some embodiments, the vascular physical examination data comprises the following parameters:

1. Visible Varicose or spider veins noted on the leg(s) and/or feet
a. Reasoning: The presence of varicose veins (enlarged, twisted veins visible under the skin) or spider veins (smaller, red or blue veins) suggests chronic venous insufficiency (CVI). In CVI, the veins in the legs have difficulty returning blood to the heart, leading to pooling of blood, which can cause the veins to become dilated and visible. These veins can also indicate an increased risk of developing other venous conditions, such as ulcers or thrombophlebitis. The physical appearance of varicose or spider veins is an important diagnostic sign of venous disease.
b. Treatment Plan:
   i. Diagnosis: Perform a duplex ultrasound to assess the severity of venous insufficiency and rule out deep vein thrombosis (DVT).
   ii. Compression Therapy: Recommend the use of medical-grade compression stockings to improve venous return, reduce swelling, and alleviate discomfort.
   iii. Lifestyle Modifications: Encourage weight management, regular physical activity (walking), and leg elevation to promote venous circulation.
   iv. Medications: Use nonsteroidal anti-inflammatory drugs (NSAIDs) for pain relief if needed. In some cases, consider medications to improve vein tone (e.g., diosmin).
   v. Procedures: If symptoms are severe, refer for sclerotherapy, laser therapy, or ambulatory phlebectomy to remove or close off varicose veins.
   vi. Monitoring: Monitor for complications like venous ulcers or thrombophlebitis, and intervene early if they arise.

2. Palpable pedal pulses
a. Reasoning: Palpable pedal pulses refer to the ability to feel the pulse in the feet and ankles (typically at the dorsalis pedis and posterior tibial arteries). A palpable pulse indicates adequate arterial blood flow to the lower extremities, suggesting good circulation. If pulses are strong and easy to detect, it generally suggests that there is no significant obstruction in the arteries and the vascular system is functioning well.
b. Treatment Plan:
   i. Diagnosis: Regular monitoring of the pulses to ensure they remain palpable and strong. No immediate intervention is necessary if pulses remain strong and symmetrical.
   ii. Health Maintenance: Emphasize maintaining healthy circulation through regular exercise, a balanced diet (low in saturated fats and high in fiber), and controlling blood pressure and cholesterol.
   iii. Patient Education: Educate the patient on lifestyle changes that support vascular health, including smoking cessation and managing comorbidities (e.g., diabetes).
   iv. Prevention: Continue to monitor for any emerging symptoms or changes that could indicate early vascular disease, even if pulses remain palpable. 3. Non-palpable pedal pulses
a. Reasoning: Non-palpable pedal pulses indicate that the pulse in the feet and ankles cannot be felt, which may be a sign of poor arterial circulation or arterial occlusion.

This could be due to conditions such as peripheral artery disease (PAD), where arteries become narrowed or blocked, reducing blood flow to the extremities. Non-palpable pulses are a warning sign of potential ischemia, where tissues may not be receiving enough oxygenated blood for proper function and healing.
b. Treatment Plan:
   i. Diagnosis: Perform an ABI (Ankle-Brachial Index) test, duplex ultrasound, or angiography to assess the extent of arterial occlusion or narrowing.
   ii. Medications: Consider prescribing antiplatelet therapy (e.g., aspirin or clopidogrel) to reduce clot formation and improve circulation.
   iii. Lifestyle Modifications: Recommend smoking cessation, a heart-healthy diet, regular walking exercises, and weight management to improve circulation and prevent further progression of PAD.
   iv. Surgical Intervention: Refer for further evaluation by a vascular surgeon if the patient has significant PAD that may require revascularization procedures (e.g., angioplasty, stenting, or bypass surgery).
   v. Monitoring: Monitor for signs of critical limb ischemia (CLI), including rest pain or non-healing ulcers, and intervene promptly if these develop. 4. Hair loss and skin changes in texture on your legs and/or feet
a. Reasoning: Hair loss and changes in skin texture on the legs and feet are common signs of poor circulation. Reduced blood flow can cause the skin to become thin, shiny, and dry, and it may lead to a loss of hair growth on the affected areas. This is often seen in patients with peripheral artery disease (PAD), where the reduced blood supply to the skin and hair follicles impairs their health. These changes can also indicate a lack of oxygen and nutrients reaching the skin, further increasing the risk of ulcers and non-healing wounds.
b. Treatment Plan:
   i. Diagnosis: Perform an ABI, duplex ultrasound, or other vascular imaging to confirm the presence of PAD and assess the severity of arterial insufficiency.
   ii. Wound Care: If skin texture changes (thin, shiny, dry) lead to ulcers or wounds, initiate appropriate wound care protocols, including debridement and the use of advanced wound care dressings.
   iii. Medications: Consider vasodilators (e.g., cilostazol) to improve circulation. Topical emollients and moisturizers can help with skin dryness and prevent further breakdown.
   iv. Lifestyle Modifications: Advise the patient on managing PAD risk factors, including controlling blood pressure, cholesterol, and blood sugar, as well as engaging in regular physical activity.
   v. Surgical Intervention: If PAD is confirmed, consider referral for revascularization procedures to improve blood flow to the affected areas and prevent further tissue damage.
   vi. Monitoring: Regular follow-up to assess skin integrity, hair regrowth, and signs of new lesions or ulcers.

5. None
a. Reasoning: This option is provided for patients who do not show any of the above physical examination findings. This helps to identify individuals who may not be exhibiting obvious signs of vascular issues at the time of the exam, but it does not rule out the presence of subclinical vascular disease or other non-physical factors affecting circulation.
b. Treatment Plan:
   i. Prevention and Health Maintenance: Encourage regular screenings (e.g., blood pressure, cholesterol, and diabetes tests) to detect early signs of vascular disease.
   ii. Lifestyle Modifications: Advise the patient on preventive measures, such as smoking cessation, weight management, regular exercise, and maintaining a healthy diet.
   iii. Monitoring: Regular check-ups to monitor for any emerging symptoms of vascular disease. Ensure that the patient is educated about recognizing early signs of PAD, varicose veins, or other vascular conditions.
   iv. Patient Education: Provide educational resources on vascular health and encourage the patient to report any new symptoms, such as leg pain, swelling, or skin changes.

In some embodiments, the patient vascular assessment data comprises real-time vascular assessment data.

In some embodiments, the patient vascular assessment data is obtained from a QuantaFlo system that is integrated with the electronic health record system.

In some embodiments, the patient vascular assessment data is obtained from Optical Character Recognition (OCR) of handwritten and printed records.

In some embodiments, the patient vascular assessment data is obtained from a third-party vascular report.

In some embodiments, the method comprises converting data from the QuantaFlo system into HL7-compliant messages to be integrated into the electronic health record system.

In some embodiments, the method comprises converting data from the QuantaFlo system into Health Level Seven (HL7)-compliant messages by a Mirth interface. HL7 is a set of standards that govern the exchange, sharing, integration, and retrieval of electronic health information. HL7 standards ensure that patient information can be shared accurately and consistently between different healthcare systems and applications. This is important because hospitals, clinics, laboratories, and other healthcare entities often use different systems.

Mirth is a prominent name in the healthcare technology landscape, particularly known for its healthcare integration engine, Mirth Connect. This open-source integration engine plays a crucial role in connecting various healthcare systems and devices. It enables seamless data exchange between different platforms, such as electronic health records (EHRs), laboratory information systems (LIS), and radiology information systems (RIS).

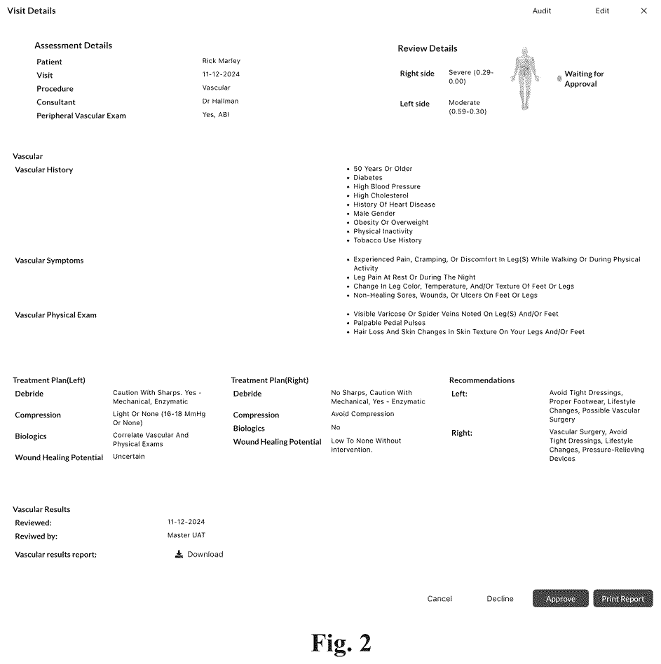

As shown in FIG. 2, the interface may include a clinical dashboard accessible to a physician or any other health care provider (e.g., nurse, health care administrator, pharmacist surgeon etc.).

In some embodiments, the clinical dashboard allows interactive modifications to the patient-specific wound care plan by a physician or any other health care provider (e.g., nurse, health care administrator, pharmacist surgeon etc.).

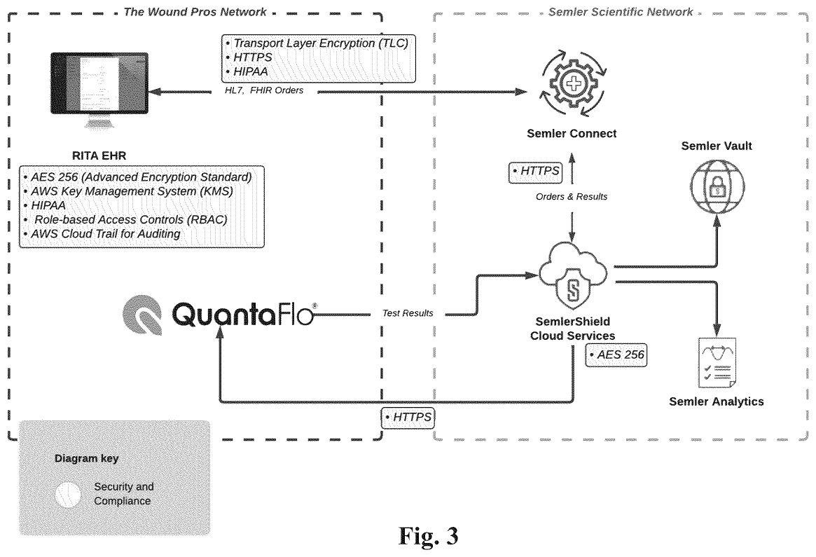

In some embodiments, the data is encrypted (as shown in FIG. 3). Certain patient information is protected by law (e.g., Healthcare Information Portability and Accountability Act (HIPAA) in the U.S.) and must be treated in a way that maintains patient privacy. Such information is termed protected health information (PHI). With respect to PHI, it is important that there is both transparency and awareness of how data entered into a mobile app is used, and that patient consent is obtained for use of PHI data. If a healthcare mobile app collects, stores, and/or transmits PHI, it is essential that it does so in full compliance with HIPAA and any other applicable laws or regulations of the country concerned. Any mobile app that is intended to connect to an Electronic Health Record (EHR) or Personal Health Record (PHR), which enables users to send and retrieve patient information between a mobile device and the EHR/PHR, must do so in a secure manner and all stakeholders involved must accept their stewardship role for protecting the PHI data contained within.

Encryption is a standard tool for ensuring the privacy of communications. A variety of encryption schemes are commercially available to secure protected information, for example the Advanced Encryption Standard (AES), promulgated by the National Institute of Standards and Technology (NIST) as Federal Information Processing Standards Publication 197, Nov. 26, 2001. AES is a symmetric encryption scheme, such that the same cipher key is used for both encoding and decoding. The AES scheme itself exists in multiple variations, such as AES counter mode, AES cipher block chaining (CBC)+cipher text stealing (CTS), RSA, and so forth. Some variations of AES may be described in Request for Comment (RFC) 3962, “Advanced Encryption Standard (AES) Encryption for Kerberos 5,” February 2005, and references cited therein.

In some embodiments, the model comprises a machine learning model. As used herein, a “machine learning model,” a “model,” or a “classifier” refers to a set of algorithmic routines and parameters that can predict an output(s) for a process input based on a set of input features, with or without being explicitly programmed. A structure of the software routines (e.g., number of subroutines and relation between them) and/or the values of the parameters can be determined in a training process, which can use actual results of the process that is being modeled. Such systems or models are understood to be necessarily rooted in computer technology, and, in fact, cannot be implemented or even exist in the absence of computing technology. While machine learning systems utilize various types of statistical analyses, machine learning systems are distinguished from statistical analyses by virtue of the ability to learn without explicit programming and being rooted in computer technology. A neural network or an artificial neural network is one set of algorithms used in machine learning for modeling the data using graphs of neurons. Any network structure may be used. Any number of layers, nodes within layers, types of nodes (activations), types of layers, interconnections, learnable parameters, and/or other network architectures may be used. Machine training uses the defined architecture, training data, and optimization to learn values of the learnable parameters of the architecture based on the samples and ground truth of training data.

A typical machine learning pipeline may include building a machine learning model from a sample dataset (referred to as a “training set”), evaluating the model against one or more additional sample datasets (referred to as a “validation set” and/or a “test set”) to decide whether to keep the model and to benchmark how good the model is, and using the model in “production” to make predictions or decisions against live input data captured by an application service. For training the model to be applied as a machine-learned model, training data is acquired and stored in a database or memory. The training data is acquired by and gation, mining, loading from a publicly or privately formed collection, transfer, and/or access. Ten, hundreds, or thousands of samples of training data are acquired. The samples are from scans of different patients and/or phantoms. Simulation may be used to form the training data. The training data includes the desired output (ground truth), such as segmentation, and the input, such as protocol data and imaging data. In some embodiments, the training set will be used to create a single classifier using any now or hereafter-known methods. In other embodiments, a plurality of training sets will be created to generate a plurality of corresponding classifiers. Each of the plurality of classifiers can be generated based on the same or different learning algorithm that utilizes the same or different features in the corresponding one of the pluralities of training sets.

Once trained, the machine-learned or trained classifier is stored for later application. The training determines the values of the learnable parameters of the network. The network architecture, values of non-learnable parameters, and values of the learnable parameters are stored as the machine-learned network. Once stored, the machine-learned network may be fixed. The same machine-learned network may be applied to different patients, different scanners, and/or with different imaging protocols for the scanning. The machine-learned network may be updated. As additional training data is acquired, such as through application of the network for patients and corrections by experts to that output, the additional training data may be used to re-train or update the training. The training is performed by optimizing parameters of the model based on outputs of the model matching or not matching corresponding labels of the first labels and optionally the second labels when the first plurality of first data structures and optionally the second plurality of second data structures are input to the model. In some embodiments, the output of the model may include a probability of being in each of a plurality of states. The state with the highest probability can be taken as the state.

In some embodiments, the machine learning model may further include a supervised learning model. Supervised learning models may include different approaches and algorithms including analytical learning, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, Nearest Neighbor Algorithm, probably approximately correct learning (PAC) learning, ripple down rules, a knowledge acquisition methodology, symbolic machine learning algorithms, subsymbolic machine learning algorithms, support vector machines, Minimum Complexity Machines (MCM), random forests, ensembles of classifiers, ordinal classification, data pre-processing, handling imbalanced datasets, statistical relational learning, or Proaftn, a multicriteria classification algorithm, linear regression, logistic regression, deep recurrent neural network (e.g., long short term memory, LSTM), Bayes classifier, hidden Markov model (HMM), linear discriminant analysis (LDA), k-means clustering, density-based spatial clustering of applications with noise (DBSCAN), random forest algorithm, support vector machine (SVM), or any model described herein.

In some embodiments, the classifier may include a supervised or unsupervised Machine Learning or Deep Learning algorithm, Logistic Regression, Naive Bayes, Support Vector Machine, Decision Tree, Random Forest, Gradient Boosting, Regularizing Gradient Boosting, K-Nearest Neighbors, a continuous regression approach, Ridge Regression, Kernel Ridge Regression, Support Vector Regression, deep learning approach, Neural Networks, Convolutional Neural Network (CNNs), Recurrent Neural Networks (RNNs), Gated Recurrent Units (GRUs), Long Short Term Memory Networks (LSTMs), Generative Models, Generative Adversarial Networks (GANs), Deep Belief Networks (DBNs), Feedforward Neural Networks, Autoencoders, Variational Autoencoders, Normalizing Flow Models, Deniosing Diffusion Probabilistic Models (DDPMs), Score Based Generative Models (SGMs), Radial Basis Function Networks (RBFNs), Multilayer Perceptrons (MLPs), Stochastic Neural Networks, or any combination thereof.

In some embodiments, the model may include a convolutional neural network (CNN). The CNN may include a set of convolutional filters configured to filter the first plurality of data structures and, optionally, the second plurality of data structures. The filter may be any filter described herein. The number of filters for each layer may be from 10 to 20, 20 to 30, 30 to 40, 40 to 50, 50 to 60, 60 to 70, 70 to 80, 80 to 90, 90 to 100, 100 to 150, 150 to 200, or more. The kernel size for the filters can be 2, 3, 4, 5, 6, 7, 8, 9, 10, 11, 12, 13, 14, 15, from 15 to 20, from 20 to 30, from 30 to 40, or more. The CNN may include an input layer configured to receive the filtered first plurality of data structures and, optionally, the filtered second plurality of data structures. The CNN may also include a plurality of hidden layers, including a plurality of nodes. The first layer of the plurality of hidden layers is coupled to the input layer. The CNN may further include an output layer coupled to a last layer of the plurality of hidden layers and configured to output an output data structure. The output data structure may include the properties.

In another aspect, this disclosure also provides a system for providing a patient-specific wound care plan for a patient based on vascular assessment, comprising one or more processors configured to implement the method as described herein.

In yet another aspect, this disclosure additionally provides a non-transitory computer-readable medium comprising instructions that, when executed by one or more processors, cause the one or more processors to perform the method as described herein.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

Aspects of the present disclosure are described herein with reference to flowchart illustrations and/or block diagrams of methods, apparatus (systems), and computer program products according to embodiments of the invention. In some embodiments, the flowchart and block diagrams in the Figures illustrate the architecture, functionality, and operation of possible implementations of systems, methods, and computer program products according to various embodiments of the present invention. In this regard, each block in the flowchart or block diagrams may represent a module, a segment, or a portion of instructions, which may include one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

These computer readable program instructions may be provided to a processor of a general-purpose computer, a special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other devices to cause a series of operational steps to be performed on the computer, other programmable apparatus, or other devices to produce a computer implemented process, such that the instructions which execute on the computer, other programmable apparatus, or other device implement the functions/acts specified in the flowchart and/or block diagram block or blocks.

It will be understood that, although the terms “first,” “second,” etc., may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of example embodiments.

Unless specifically stated otherwise, as apparent from the above discussion, it is appreciated that throughout the description, discussions utilizing terms such as “processing,” “performing,” “receiving,” “computing,” “calculating,” “determining,” “identifying,” “displaying,” “providing,” “merging,” “combining,” “running,” “transmitting,” “obtaining,” or the like, refer to the action and processes of a computer system, or similar electronic computing device, that manipulates and transforms data represented as physical (or electronic) quantities within the computer system memories or registers or other such information storage, transmission or display devices.

As used herein, the term “if may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “classifiers” refers generally to various types of classifier frameworks, such as neural network classifiers, hierarchical classifiers, ensemble classifiers, etc. In addition, a classifier design can include a multiplicity of classifiers that attempt to partition data into two groups, either organized hierarchically or run in parallel, and then combined to find the best classification. Further, a classifier can include ensemble classifiers wherein a large number of classifiers all attempting to perform the same classification task are learned, but trained with different data/variables/parameters, and then combined to produce a final classification label. The classification methods implemented may be “black boxes” that are unable to explain their prediction to a user (which is the case if classifiers are built using neural networks, for example). The classification methods may be “white boxes” that are in a human-readable form (which is the case if classifiers are built using decision trees, for example). In other embodiments, the classification models may be “gray boxes” that can partially explain how solutions are derived (e.g., a combination of “white box” and “black box” type classifiers).

As used herein, the term “classification” refers to any number or other characters that are associated with a particular property of a sample. The classification can be binary (e.g., positive or negative) or have more levels of classification (e.g., a scale from 1 to 10 or 0 to 1). The term “cutoff” or “threshold” refers to a predetermined number used in an operation. For example, a cutoff value can refer to a classification score as used above. A threshold value may be a value above or below which a particular classification applies. Either of these terms can be used in either of these contexts.

The terms or acronyms like “convolutional neural network,” “CNN,” “neural network,” “NN,” “deep neural network,” “DNN,” “recurrent neural network,” “RNN,” and/or the like may be interchangeably referenced throughout this document.

An “electronic device” or a “computing device” refers to a device that includes a processor and memory. Each device may have its own processor and/or memory, or the processor and/or memory may be shared with other devices as in a virtual machine or container arrangement. The memory will contain or receive programming instructions that, when executed by the processor, cause the electronic device to perform one or more operations according to the programming instructions.

The terms “processor” and “processing device” refer to a hardware component of an electronic device that is configured to execute programming instructions, such as a microprocessor or other logical circuit. A processor and memory may be elements of a microcontroller, custom configurable integrated circuit, programmable system-on-a-chip, or other electronic device that can be programmed to perform various functions. Except where specifically stated otherwise, the singular term “processor” or “processing device” is intended to include both single-processing device embodiments and embodiments in which multiple processing devices together or collectively perform a process.

In this document, the terms “communication link” and “communication path” mean a wired or wireless path via which a first device sends communication signals to and/or receives communication signals from one or more other devices. Devices are “communicatively connected” if the devices are able to send and/or receive data via a communication link. “Electronic communication” refers to the transmission of data via one or more signals between two or more electronic devices, whether through a wired or wireless network, and whether directly or indirectly via one or more intermediary devices.

The terms “memory,” “memory device,” “computer-readable medium,” “data store,” “data storage facility” and the like each refer to a non-transitory device on which computer-readable data, programming instructions or both are stored. Except where specifically stated otherwise, the terms “memory,” “memory device,” “computer-readable medium,” “data store,” “data storage facility” and the like are intended to include single device embodiments, embodiments in which multiple memory devices together or collectively store a set of data or instructions, as well as individual sectors within such devices.

The terms “processor” and “processing device” refer to a hardware component of an electronic device that is configured to execute programming instructions, such as a microprocessor or other logical circuit. A processor and memory may be elements of a microcontroller, custom configurable integrated circuit, programmable system-on-a-chip, or other electronic device that can be programmed to perform various functions. Except where specifically stated otherwise, the singular term “processor” or “processing device” is intended to include both single-processing device embodiments and embodiments in which multiple processing devices together or collectively perform a process.

In this document, the terms “communication link” and “communication path” mean a wired or wireless path via which a first device sends communication signals to and/or receives communication signals from one or more other devices. Devices are “communicatively connected” if the devices are able to send and/or receive data via a communication link. “Electronic communication” refers to the transmission of data via one or more signals between two or more electronic devices, whether through a wired or wireless network, and whether directly or indirectly via one or more intermediary devices.

It is noted here that, as used in this specification and the appended claims, the singular forms “a,” “an,” and “the” include plural reference unless the context clearly dictates otherwise.

As used herein, “plurality” means two or more. As used herein, a “set” of items may include one or more of such items.

As used herein, “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like do not necessarily refer to the same embodiment, but may unless the context dictates otherwise.

As used herein, the terms “and/or” or “/” means any one of the items, any combination of the items, or all of the items with which this term is associated.

As used herein, the term “substantially” does not exclude “completely,” e.g., a composition which is “substantially free” from Y may be completely free from Y. Where necessary, the word “substantially” may be omitted from the definition of the present disclosure.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As used herein, the term “each,” when used in reference to a collection of items, is intended to identify an individual item in the collection but does not necessarily refer to every item in the collection. Exceptions can occur if explicit disclosure or context clearly dictates otherwise.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

The use of any and all examples, or exemplary language (e.g., “such as”) provided herein, is intended merely to better illuminate the invention and does not pose a limitation on the scope of the present disclosure unless otherwise claimed. No language in the specification should be construed as indicating any non-claimed element as essential to the practice of the present disclosure.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present disclosure is not entitled to antedate such publication by virtue of prior disclosure. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### Examples

The disclosed methods and systems involve automatically analyzing vascular exams, categorizing patients into specific brackets within the database, and generating tailored treatment guidance based on their vascular results. RITA is an Electronic Medical Record (EMR) system. Vascular in RITA is the digital platform that will hold all vascular data in each individual patient card. The platform includes: RITA EHR system, Semler's QuantaFlo diagnostic tool, Clinician Questionnaire, and Automated Ordering System (HL7/Mirth Integration). In some embodiments, the process includes: (1) a user (e.g., clinician) accesses patient's card in RITA; (2) adds ‘Vascular’ section; (3) selects QuantaFlo as the vascular procedure; (4) completes the clinician questionnaire; and (5) generates the Vascular Plan of Care based on QuantaFlo results.

The methods and systems integrate Semler's QuantaFlo seamlessly into RITA. Clinicians initiate QuantaFlo assessments via the RITA interface. Patient data and assessment requests are automatically transmitted to SemlerConnect using HL7/Mirth Integration. QuantaFlo results are received, processed, and presented within RITA's ‘Vascular’ section.

A digital request will be transmitted to Semler Scientific through the HL7/Mirth integration, indicating the necessity for a patient's vascular examination. This request triggers SemlerConnect's HL7 and Mirth digital program, extracting the specific patient data from RITA. Subsequently, the data is transferred to Semler Scientific's Quantaflo software. After the clinician completes and saves the vascular examination, SemlerConnect stores the Vascular report securely in the cloud. SemlerConnect then transfers the patient's specific vascular report into RITA, placing it in the appropriate patient card under the Vascular tab. Upon receiving the vascular report, an automated OCR scanner reviews the data and generates a customized treatment plan, outlining aspects like healing, debridement, compression, biologics, and consultations. Essentially, this process creates a tailored treatment plan for each patient based on their individual needs.

The disclosed methods and systems (also herein referred to as “Integrated Vascular Assessment System” or “IVAS”) utilize automation to remove human error to create an individualized plan of care based on a patient's vascular exam. The methods and systems solve the problem of creating a focused, individualized treatment plan based on a set of specific parameters provided by the vascular exam. The disclosed methods and systems are special/unique compared to other similar existing methods because they create an automated step to create a treatment plan that will help guide care for patients. This will benefit patients, families, clinicians, agencies, and other stakeholders in patients' care. IVAS is capable of streamlining vascular assessments, reducing manual data entry, enhancing accuracy, and ensuring timely patient care. It solves the problem of integration complexity between different healthcare technologies, ensuring a secure and efficient workflow for clinicians. IVAS stands out due to its seamless integration approach, ensuring data accuracy, security, and user-friendliness. Its ability to incorporate QuantaFlo data directly into the electronic health record (EHR) system makes it unique compared to other standalone diagnostic tools.

The system integrates the artificial intelligence (AI)-powered EHR system, the RITA Application. RITA does not just manage patients; it cares for them like family. It does not simply provide wound care treatment protocols; it redefines them. It does not just measure wounds; it anticipates their progress.

Semler: Semler is a healthcare technology company known for its product, ‘SemlerConnect’, which is an electronic medical record (EMR) integration engine facilitating effective healthcare data exchange. Semler Scientific also offers QuantaFlo, a diagnostic tool for vascular assessment. The use of Quantaflo in wound care is new.

Utilization of QuantaFlo: The utilization of QuantaFlo brings the following benefits: (a) It aligns with the American Heart Association's recommendation for testing individuals at risk of peripheral arterial disease, including diabetics, those with a history of smoking, heart disease, kidney disease, hypertension, hypolipidemia, and anyone aged 65 or older. QuantaFlo is the ideal tool for conducting these assessments; (b) As we employ biologics for advanced wound care, it's essential to maintain vascular reports. QuantaFlo serves this requirement seamlessly; and (c) Most importantly, the core mission revolves around healing wounds, and blood plays a vital role in this process. By gaining a comprehensive understanding of each patient's vascular status through QuantaFlo, the treatment protocols can be tailored more effectively.

In summary, QuantaFlo not only aids in complying with medical recommendations but also enhances our ability to provide optimal care and healing for our patients.

QuantaFlo is a new diagnostic tool for vascular assessment, not yet implemented in the system. The problem includes: (a) Integration Complexity: Integrating a new technology like QuantaFlo with our existing EHR system poses challenges due to differences in data formats and workflows; (b) Data Fragmentation: Without integration, QuantaFlo data will remain separate from RITA, potentially leading to errors and inefficiencies; and (c) Patient Care Delays: Manual data entry for QuantaFlo tests can lead to delays in patient care, impacting healthcare providers' ability to make timely decisions.

The project aims to achieve the following goals: (a) Seamless Integration: Implement a smooth and reliable integration of Semler's QuantaFlo feature within RITA; (b) Efficiency: Eliminate manual data entry by automating the ordering and result retrieval process for QuantaFlo tests; (c) Accuracy: Ensure accurate and timely transfer of patient information and QuantaFlo test results; and (d) Security: Maintain a secure environment for healthcare data exchange, adhering to industry standards.

### Areas of System Affected

| Area of System | |
|---|---|
| Affected | Comments |
| Appointment Module | Integration with the appointment scheduling system |
| | for trigger events related to QuantaFlo assessments. |
| Wound Evaluation | Modification to the ‘wounds’ section to include |
| | ‘Vascular’ as an option |
| Questionnaire | Development of a questionnaire for clinicians related |
| | to QuantaFlo assessment. |
| HL7/Mirth Integration | Integration with SemlerConnect for order transmission. |

### Actors

| Actor | Comments |
|---|---|
| Clinicians | The primary users, responsible for initiating QuantaFlo |
| | assessments during patient appointments. |
| Billing Team | Responsible for billing |
| Medical Records Team | Intermediary in assisting with current vascular reports being |
| | placed in the appropriate “Vascular” tab within RITA. |
| | Rectify errors when they occur. |
| | (e.g., A clinician reports they performed a QuantaFlo and |
| | the questionnaire is filled out, but there is no vascular report. |
| | MR can alert of this error) |
| SemlerConnect Services | The intermediary server for initiating QuantaFlo assessments |
| | during patient appointments. |
| RITA Integration Engine | Part of the RITA system responsible for communicating |
| | with SemlerConnect via HL7/Mirth. |

### Non-Functional Requirements

| Non-functional | |
|---|---|
| requirement category | Non-functional requirement |
| Software | The feature will run on PHP Back-End and Front- |
| | End: React (Web), Kotlin (Android), Swift (IOS) |
| User Experience | The user interface should be intuitive and user- |
| | friendly, allowing users to easily navigate and |
| | accessible to clinicians, and other departments |
| | involved. |
| Scalability | The system shall be scalable to accommodate an |
| | increasing number of QuantaFlo assessments as |
| | the user base grows. |
| Reliability | The system shall queue orders and result in the |
| | event of network disruptions to ensure data |
| | integrity. |
| | The system shall provide priority support with |
| | alerting and monitoring to address issues |
| | promptly. |
| Security | SSO integration |
| | The system shall enable Two-Factor |
| | Authentication (2FA). |
| | The system shall ensure TLS 1.2 encryption for |
| | HTTPS communication with SemlerConnect. |
| | The system shall comply with industry standards |
| | for data security and privacy. |
| Compatibility | The application shall ensure cross browser |
| | compatibility (chrome, Safari, Mozilla etc.) |
| Usability | The system shall ensure a user-friendly |
| | interface for clinicians to complete QuantaFlo |
| | assessments. |
| Flexibility | The system shall support HL7, FHIR, or |
| | customized data formats as required by Semler |
| | Connect. |
| | The system shall allow for customization of |
| | QuantaFlo app settings through SemlerShield. |

### Functional Requirements

| User_Story | Requirements |
|---|---|
| As a Clinician, I want to seamlessly | The system shall integrate Semler's QuantaFlo |
| integrate Semler's QuantaFlo | feature seamlessly with the RITA EHR system. |
| feature with our RITA EHR system | The system shall modify the “Wounds” section of |
| So that I can efficiently order and | RITA to include “Vascular” as an option for future |
| retrieve QuantaFlo test results | QuantaFlo assessments. |
| without manual data entry. | The system shall develop and incorporate a |
| | questionnaire for clinicians to gather relevant |
| | patient information before QuantaFlo assessments. |
| | The system shall trigger an order to SemlerConnect |
| | via HL7/Mirth when clinicians indicate the need |
| | for a QuantaFlo assessment. |

### Inform: Vascular Screening

All agencies will receive notification of vascular testing.

The sales department will include Quantaflo in-service to inform all new agencies.

Patient Navigators (PN) will add verbiage when confirming appointments about the upcoming vascular exam.

Clinicians will update Case Managers (CM) during their weekly updates on their patients.

A mass email will be sent to all current agencies about the vascular testing.

### Trigger:

An automatic trigger event will be placed on each active patient, automatically reactivated yearly.

The trigger event will send an order to Semler via HL7/MIRTH to indicate an upcoming vascular test.

Upcoming orders may experience a 1-2 week delay from initiation.

The order trigger will involve Semler pulling patient data from RITA and pushing data back into Quantaflo software, requiring internet connectivity.

Semler recommends disabling manual input of patient data to prevent errors and ensure the vascular report is placed in the correct chart.

Clinicians are recommended to open Quantaflo at home with internet access at night to allow data transfer.

Semler reports that 1700 orders are well within performance limits on their end, with a daily load of 300-400 patients.

Per Myla, the average number of patients seen per day is 340, with 2068 active patients.

### Patient's Card:

Log in to RITA and enter the patient's card. Access patients from the list of scheduled appointments by selecting the icon on the left-hand side and opening the appropriate patient by clicking on their name.

### Vascular Procedure:

Click on the red “+” sign to select the correct vascular procedure or implement a drop-down menu for clinicians to choose between Quantaflo and ABI under the “Vascular” section in “Appointment Details.”

Select “Create” or choose the procedure from the drop-down menu, which will auto-populate two assessments: Vascular Questionnaire and Vascular Plan of Care.

### Vascular Questionnaire:

Click on “Start Assessment” to begin the Vascular Questionnaire.

Refer to the questionnaire link:

### Update: Once Per Year

After completing the questionnaire, click “Create Assessment” to save it.

The Vascular Questionnaire should be conducted face-to-face with the patient.

The saved questionnaire should be transformed into a report similar to the provided example.

### Vascular Questionnaire

Did you perform a Quantaflo/ABI Today? Yes Medical History:
Do you have a history of diabetes? Yes
Have you been diagnosed with high blood pressure (hypertension)? Yes
a. If yes. are taking medication to manage your high blood pressure? Yes. Lisinopril
Have you ever been told you have high cholesterol? Yes
a. If yes. are taking medication to manage your high cholesterol? Yes, atorvastatin
Have you been diagnosed with heart disease or had any heart-related procedures? Yes a. If yes. are you taking heart medication? Yes, metoprolol
Are you a current or former smoker? Yes or Do you use nicotine products? Yes. gum
If yes, was smoking/nicotine cessation discussed? Yes
Patients who currently smoke or who have smoked within the past 6 weeks are not candidates for biologies.
Do you have a history of kidney disease or dialysis? Yes, CKD3
Have you ever had blood clots or been diagnosed with deep vein thrombosis (DVT)? No
Have you had any previous vascular surgeries or procedures? No
Are you taking any blood thinner medications (ie. Aspirin)? Yes a. If yes, please provide details: Aspirin
Do you have a family history of vascular diseases or circulatory problems? Yes
Do you have a history of amputation(s)? Yes a. If yes, please provide details. Left great toe 2001
Do you have a history of gangrene? Yes
a. If yes, please provide details: Left great toe 2001
Symptoms:
Have you experienced pain, cramping, or discomfort in your legs while walking or during physical activity? Yes
a. If yes. please describe the location and nature of the pain (Intermittent Claudication): painful cramp after 2 blocks
Do you experience leg pain at rest or during the night (rest pain)? No
Have you noticed any changes in the color, temperature, or texture of your feet or legs? Yes
Are there any non-healing sores, wounds, or ulcers on your feet or legs? Yes
Have you observed any swelling in your legs, ankles, or feet? Yes
Do you feel numbness, tingling, or a sensation of “pins and needles” in your feet or legs? Yes
Physical Examination:
Please rate your ability to walk without pain on a scale of 0 to 10 (0=no pain, 10=severe pain) 8.
Are there any visible varicose veins or spider veins on your legs? No
Please describe any areas of tenderness or pain in your legs. Bilateral entire legs
Are your leg pulses (such as the pulse in your foot or behind the knee) easily palpable? No
Have you noticed any hair loss or changes in skin texture on your legs or feet? Yes, no hair visible below knee bilaterally
Can you wiggle your toes and move your feet freely without difficulty? No
The report can be generated by clicking on vertical dots.
It can also be found in the Patient's card under “Encounter List,” then under the sub-heading “Vascular.”
All vascular reports should use the Global Wound Care Medical Group logo and billing demographics per the Billing department.

### Vascular Plan of Care:

Suggest a solution if a problem is identified, as per Medicare requirements.

Optical Character Recognition (OCR) will read the Quantaflo/ABI report to make recommendations, which are color-coded and risk-stratified.

Vascular Plan of care based on the above documents.

The primary goal of every patient is complete wound closure. Peripheral Arterial Disease (PAD) Testing was performed to develop an individual care plan to guide wound care treatment. Based on the results of the PAD test, it was determined that the patient has (high, moderate, range of uncertainty=questionable, low) healing potential. It has been determined that (sharp, mechanical, enzymatic) wound debridement (can, cannot) be performed (safely, with caution). If applicable, (high 40-50 mmHg 4-layer, moderate 25-35 mmHg 3-layer, mild compression 16-18 mmHg 1-2 layers, no) compression can be applied. The use of advanced wound care biologics (can, cannot) be applied. Recommendations for the patient also include (wear properly fitted footwear, appropriate offloading and pressure-relieving devices, lifestyle changes) (Wear properly fitted footwear, appropriate offloading and pressure-relieving devices, lifestyle changes, and medication) (Avoid tight dressings, wear properly fitted footwear, appropriate offloading and pressure-relieving devices, lifestyle changes, medication, vascular consult to explore methods of improving blood flow) (Avoid tight dressings, wear properly fitted footwear, appropriate offloading and pressure-relieving devices, lifestyle changes, medication, vascular consult to explore methods of improving blood flow). The patient will undergo PAD testing at a minimum once per year.

Medication recommendations: The patient may benefit from (antiplatelet, statin, antihypertensive, glycemic control) agents to reduce the risk of Myocardial Infarction, stroke, vascular death, heart failure.

### American Heart Association Recommendations

Smoking History: The patient (does, does not) have a smoking history. Smoking cessation: Patients with PAD who smoke cigarettes or use other forms of tobacco are advised to quit to improve circulation and wound healing potential.

Physical Activity: An effective treatment for PAD symptoms is regular physical activity. Supervised exercise therapy (SET) is advised.

### ABI and 3^{rd} Party Vascular Reports

Currently, there is no place to put the ABI report or vascular reports from 3^{rd}-parties. To correct this, I propose that when you enter the patient's full patient card by going to the map, and searching for the patient's name, and selecting the patient. In the patient's card, click on the heading tab “Encounter List” and the sub-tab heading “Vascular.” This is the area in which Semler will push the Quantaflo report. This area is to access all vascular reports: Vascular Report: Quantaflo, ABI, or 3^{rd} party, Vascular Questionnaire, and The Vascular Plan of Care. All reports should be printable from this area. This area should also emulate the documents tab to allow the user to manually upload a Quantaflo, ABI, or 3^{rd}-party vascular report, or can upload by clicking on the red “+” sign.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method for providing a patient-specific wound care plan for a patient based on vascular assessment, comprising:
receiving patient vascular assessment data of the patient, wherein the patient vascular assessment data comprises vascular history data, vascular symptom data, and vascular examination data;
processing and inputting the patient vascular assessment data to an electronic health record system;
analyzing the patient vascular assessment data by a trained model;
generating, by the trained model, a patient-specific wound care plan for the patient based on the vascular history data, the vascular symptom data, and the vascular examination data;
populating the patient-specific wound care plan within the electronic health record system; and
presenting the patient vascular assessment data and the patient-specific wound care plan on an interface accessible to a heath care provider.

2. The method of claim 1, wherein the vascular history data comprises one or more of: age 50 years or older, African American ethnicity, currently on blood thinner, diabetes, elevated homocysteine levels, family history of vascular disease, history of amputations, high blood pressure, high cholesterol, history of gangrene, history of heart disease, inflammatory conditions, kidney disease, male gender, obesity or overweight, open wound, physical inactivity, tobacco use history, stroke, and prior vascular health history.

3. The method of claim 2, wherein the vascular symptom data comprises one or more of: experienced pain, cramping, or discomfort in leg(s) while walking or during physical activity; leg pain at rest or during the night; change in leg color, temperature, and/or texture of feet or legs; non-healing sores, wounds, or ulcers on the feet or legs; swelling in leg(s), ankle(s), and/or feet; numbness, tingling, or sensations of pins and needles in your feet and/or legs; and none.

4. The method of claim 1, wherein the vascular physical examination data comprises one or more of: visible varicose or spider veins noted on the leg(s) and/or feet; palpable pedal pulses; non-palpable pedal pulses; hair loss and skin changes in texture on your legs and/or feet; and none.

5. The method of claim 1, wherein the patient vascular assessment data comprises real-time vascular assessment data.

6. The method of claim 1, wherein the patient vascular assessment data is obtained from a QuantaFlo system that is integrated with the electronic health record system.

7. The method of claim 1, wherein the patient vascular assessment data is obtained from Optical Character Recognition (OCR) of handwritten and printed records.

8. The method of claim 1, wherein the patient vascular assessment data is obtained from a third-party vascular report.

9. The method of claim 6, comprising converting data from the QuantaFlo system into HL7-compliant messages to be integrated into the electronic health record system.

10. The method of claim 9, comprising converting data from the QuantaFlo system into HL7-compliant messages by a Mirth interface.

11. The method of claim 9, wherein the data is encrypted.

12. The method of claim 1, wherein the interface comprises a clinical dashboard accessible to a clinician.

13. The method of claim 12, wherein the clinical dashboard allows interactive modifications to the patient-specific wound care plan by the clinician.

14. The method of claim 1, wherein the trained model comprises a machine learning model.

15. The method of claim 14, wherein the machine learning model comprises a supervised or unsupervised machine learning model.

16. The method of claim 14, wherein the machine learning model comprises Deep Learning algorithm, Logistic Regression, Naive Bayes, Support Vector Machine, Decision Tree, Random Forest, Gradient Boosting, Regularizing Gradient Boosting, K-Nearest Neighbors, a continuous regression approach, Ridge Regression, Kernel Ridge Regression, Support Vector Regression, deep learning approach, Neural Networks, Convolutional Neural Network (CNNs), Recurrent Neural Networks (RNNs), Gated Recurrent Units (GRUs), Long Short Term Memory Networks (LSTMs), Generative Models, Generative Adversarial Networks (GANs), Deep Belief Networks (DBNs), Feedforward Neural Networks, Autoencoders, Variational Autoencoders, Normalizing Flow Models, Deniosing Diffusion Probabilistic Models (DDPMs), Score Based Generative Models (SGMs), Radial Basis Function Networks (RBFNs), Multilayer Perceptrons (MLPs), Stochastic Neural Networks, or a combination thereof.

17. A system for providing a patient-specific wound care plan for a patient based on vascular assessment, comprising one or more processors configured to implement the method of claim 1.

18. A non-transitory computer-readable medium comprising instructions that, when executed by one or more processors, cause the one or more processors to perform the method of claim 1.
